(19) United States
(12) Patent Application Publication
(10) Pub. No.: US 2006/0134186 A1
(43) Pub. Date: Jun. 22, 2006

(54) Oxygenating agents for enhancing host responses to microbial infections

(76) Inventors: Richard M. Carlton, Port Washington, NY (US); Jeffrey E. Galpin, Westlake Villages, CA (US)

Correspondence Address: MORGAN LEWIS & BOCKIUS LLP, 1111 PENNSYLVANIA AVENUE NW, WASHINGTON, DC, 20004, US

(21) Appl. No.: 10/509,285

(22) PCT Filed: Mar. 26, 2003
(86) PCT No.: PCT/US03/09226

§ 371 Date: Jun. 27, 2005

Related U.S. Application Data
(60) Provisional application No. 60/367,732, filed on Mar. 28, 2002.

(51) Int. Cl. A61K 38/42; A61K 9/70; A61K 9/127

(52) U.S. Cl. 424/449; 514/6; 424/450

(57) ABSTRACT

Oxygenating agents are used in the present invention for the novel purpose of treating microbial infections. The invention takes advantage of the fact that the increase in tissue pO₂ produced thereby can enhance the efficacy of the body's own antimicrobial defenses (including tissue repair), while also improving the efficacy of adjunctive agents that may be co-administered, such as antimicrobial agents, antioxidants, cytokine modulators, endotoxin antagonists and growth factors. The oxygenating agents of the present invention achieve the desired increased pO₂ in the tissues without having to resort to the risks and expenses of hyper-baric oxygen therapy (HBO). The oxygenating agents can be administered systemically, regionally, or topically.

### BACKGROUND OF THE INVENTION

1. Field of the Invention

The present invention relates to the use of oxygenating agents that, while well known in the art, are used herein in a novel application to enhance the host responses to infections, as well as to improve the in vivo efficacy of antimicrobial agents directed against infections in ischemic tissues (where low oxygen tension and other local conditions tend to impair the efficacy of said antibiotics).

2. Description of the Related Art

When infections are difficult to treat, whether because of (i) multidrug resistance to the antimicrobial agents, (ii) poor host defenses (as in AIDS), (iii) rapidly multiplying and rapidly spreading infections (as in necrotizing fasciitis), (iv) ischemia or hypoxia that is causing antimicrobial efficacy to be reduced (and increasing the ability of various microbes to multiply and spread), or for other reasons, the outcome is generally poor. For example, there will typically be an increased loss of limb, bone or tissue (e.g. through amputations of gangrenous bed sores, where there also can exist myonecrosis, myofasciitis, or acute or chronic osteomyelitis); an increased loss of life (through the opportunity of protracted infections to reach and spread in the bloodstream); and increased costs to the health care system. The increased health care costs are due to factors such as longer hospital stays; the surgery required for debridement of infected tissue and bone; or for plastic reconstructive surgery; the increased risk that these patients will develop complications such as recurrent sepsis, ARDS (Adult respiratory distress syndrome), renal or heart failure, and DIC (disseminated intravascular coagulation); the expensive combination regimens of antimicrobials that must be tried; and finally, the long term convalescence and complications and debility resulting from prolonged bed rest (such as pulmonary emboli, pneumonia, osteoporosis, and additional bed sores).

In all the difficult types and sites of infections listed above, increasing the pO₂ at the infected site can generally help in the cure of the infection; because:

(i) In the case of multidrug resistant and/or rapidly multiplying microbes, an increase in pO₂ is known to have “static” and “cidal” effects on a wide variety of such microbes (including bacteria and fungi).

(ii) In the case of poor host defenses, an increase in pO₂ is known to be able to improve host defenses (e.g. by increasing oxidative bursts that are harmful to intracellular or extracellular pathogens.)

(iii) Where hypoxia interferes with the efficacy of antimicrobial agents, an increase in pO₂ can overcome the conditions that are interfering with said efficacy. For example, it is well known that the low pO₂ of hypoxic sites induces bacteria to decrease their rate of replication, so that a subsequent increase in pO₂ induces those bacteria to multiply, at which time they then become susceptible to the many cell wall-acting antibiotics whose mechanism of action requires the target bacteria to be actively multiplying.

It is thus apparent that an increase in pO₂ would assist in the treatment of infections from a wide variety of pathogens. However, the only method known in the art for increasing pO₂ in order to treat infections is the use of hyperbaric oxygen (“HBO”).

Experimental evidence in animals and/or in humans has established the following principles concerning HBO therapy of infections.

1. It enhances wound closure and tissue repair by (a) causing proliferation of fibroblasts and capillaries, (b) reducing edema, (c) reducing acidosis, and (d) producing microvascular neoangiogenesis. [See e.g. Elliott D C, Kufera J A, Myers R A. Necrotizing soft tissue infections: Risk factors for mortality and strategies for management. Ann Surg 1996 November; 224(5): 672-83.]

2. It enhances host immunity by (a) stimulating oxygen burst, (b) stimulating free radical formation, (c) restoring proper redox potential, (d) promoting the ability of polymorpho-nuclear cells to complete their metabolic pathways, including those that are directed against microbes, (e) regulating cytokine and chemokine dynamics, and (f) reducing lactic acidosis. [See e.g. Shafer M R. Use of hyperbaric oxygen as adjunct therapy to surgical debridement of complicated wounds. Semin Perioper Nurs 1993 October; 2(4):256-62.]

3. It directly interferes with many pathogens, by (a) direct bactericidal, virucidal or fungicidal actions and by (b) reducing the amount of toxin released by the pathogen, as for example the ability of HBO to reduce the release of clostridial alpha toxins. The evidence for an antiviral effect of HBO is found in the report by Altiere et al., wherein HBO decreased the load of HIV virus in peripheral blood mononuclear cells, and wherein few HIV viruses entered uninfected cells that had been exposed to HBO. [See: (a) Altieri R J. HIV antiviral effects of hyperbaric oxygen therapy. J. Assoc Nurses AIDS Care 1996 January-February; 7(1):43-5, and (b) Kajs-Wyllie M. Hyperbaric oxygen therapy for rhinocerebral fungal infection. J Neurosci Nurs 1995 June; 27(3):174-81.]

4. Finally, HBO aids certain tissues (such as the gut wall) to resist microbial invasion into sterile areas of the body (such as the bloodstream), such invasion being far more likely to occur where hypoxic conditions prevail.

However, there are many risks with and drawbacks to using hyperbaric oxygen therapy. One of the drawbacks is that hyperbaric chambers are costly and require large dedicated areas, so that HBO is not available in most secondary and tertiary hospital centers, let alone in doctor's offices. Furthermore, even when HBO is available, the high oxygen tensions that are produced throughout the body can generate oxygen free radicals in delicate tissues such as the interior of the eye, resulting in cataracts or other undesired sequelae. Furthermore, HBO is known to cause toxicity to the central nervous system (seizures being one of such symptoms) as well as to the lungs (decompression illness), and to disturb equilibrium in the ear (requiring myringotomy in some cases). In addition, HBO may not succeed in penetrating all compartments equally, such as the bowel or the interior of specific hollow organ sites. Finally, since most infected ischemic tissues would require multiple treatments over a number of days or weeks, the risks to the patient and the costs to the healthcare system accumulate progressively with the number and frequency of such treatments.

Thus, since there is a compelling need to raise the tissue pO₂ in treating a wide variety of microbial infections, an alternative to HBO would be of great value. Oxygenating agents, which have been developed to overcome ischemia in various tissues, would be a logical alternative, but they have not been administered to treat infected tissues. [For examples of the use of oxygenating agents to treat ischemic conditions, see, for example, Iwai et al. “A new treatment for ischemic foot bath therapy using oxygen soluble fluid”, J. Cardiov. Surg. 30:490-493, 1989; Waxman et al. “Perfluorocarbons as blood substitutes” Ann. Emerg. Med., 15:1423-1424, 1986; U.S. Pat. No. 4,795,423 “Oxygenated perfluorinated perfusion of the ocular globe to treat ischemic retinopathy”; and Tur et al. “Topical hydrogen peroxide treatment of ischemic skin ulcers in the guinea pig: Blood recruitment in multiple skin sites”, J. of the Amer. Acad. of Dermat; 33:217-221, 1995.]

In all the references cited, one or another oxygenating agent was applied, whether topically (onto superficial ischemic tissues ) or injected directly into ischernic tissues (e.g. in the case of the U.S. Patent cited above concerning the ocular globe in cases of retinopathy). While one of the oxygenating agents mentioned in the references just cited—hydrogen peroxide—has been used to treat infections as well as to treat ischemia, nevertheless, as will be discussed in detail below, the type of oxygenating agent exemplified by hydrogen peroxide does not penetrate tissues and so can only be used topically and for the most superficial infections. However, the prior art has not taught the administration of penetrating oxygenating agents to tissues that are infected. In the present invention, tissues that are infected (whether or not ischemic) are treated with penetrating oxygenating agents in order (i) to enhance the efficacy of the host's reparative processes and of its antimicrobial defenses, and (ii) to improve the efficacy of antimicrobial agents. The present invention thus provides an alternative to systemic HBO therapy of infections.

### SUMMARY OF THE INVENTION

Oxygenating agents that are known in the art are used by the present invention in a new application, for the novel purpose of treating microbial infections. The invention takes advantage of the fact that the increase in tissue pO₂ produced thereby can enhance the efficacy of the body's own antimicrobial defenses while also promoting wound repair, and at the same time improving the efficacy of antimicrobial agents that may be prescribed. The oxygenating agents can be administered systemically, but they can also be administered regionally, that is, to specific tissues, without toxicity to other regions (such as the cornea) that may be harmed by an increased pO₂. In either case, the oxygenating agents are used in an effective amount to achieve the required Eh levels in infected tissues.

Some oxygenating agents are already approved for commercial sale, but these approvals are for non-infectious indications, namely and primarily (i) the treatment of ischemia, and (ii) the replacement of blood lost in trauma or in elective surgery.

The present invention is preferably practiced by co-administering an antimicrobial agent known to kill or attenuate the microbe of interest (e.g., a bacterium, fungus, yeast, parasite, virus, or any other microorganism causing an infection ), in combination with at least one oxygenating agent. If the antimicrobial agent and oxygenating agent are co-administered for synergy, they can either be administered together in the same pharmaceutical preparation, or separately in time and in space (by different routes, e.g., one topically and the other intravenously). Increasing the pO₂ in the infected tissue allows the efficacy of the antimicrobial agent to approximate the level it would have in normal (i.e., atmospheric) or above-normal ranges of oxygen tension. The present invention can thereby achieve synergy among host defenses, immunity, and antibiotics. In any case, the co-administration of the antimicrobial agent is not necessary for the practice of the invention.

There are several reasons why antimicrobials tend to have poor efficacy in hypoxic/ischemic tissues. 1) For many antimicrobials to work, the target pathogen must be actively multiplying. However, such replication is considerably inhibited by reduced oxygen tension. That is why bacterial growth rate is diminished late in the course of chronic suppurative infection, and that in turn explains why the bacteria become refractory to antibiotic therapy. 2) A second reason for the greatly reduced efficacy of antimicrobials in infected ischemic/hypoxic tissues is that such tissues tend to have an acidic milieu. This is due in part to the hypoxia, and, in part, to the sequelae of the infection/inflammation itself. Such acidic milieus, with their low redox potential, inhibit the action of certain antimicrobials (e.g., aminoglycosides). That is why alkalinizing agents are commonly used to restore the efficacy of certain antibiotics, such as erythromycin, lincomycin, clindamycin, and the aminoglycosidic aminocyclitol antibiotics. 3) A third reason for the reduced efficacy is that the penetration of certain antimicrobials into the target pathogen requires an oxygen-dependent step, and such entry therefore becomes inhibited by low oxygen tensions.

It is contemplated that antioxidants (such as superoxide dysmutase (“SOD”), tocopherol, and ascorbic acid), growth factors, endotoxin antagonists, cytokine modulators, and numerous other synergizing agents can also be co-administered with the oxygenating agents of the present invention, in order to protect against any free radicals that might be engendered by (i) the respiratory oxidative stress created by certain infections (such as the influenza virus), as well as by (ii) the oxygenating agents of the present invention themselves. Synergizing agents, such as the ones listed, can also promote more rapid healing of wounds; can counter the actions of various pro-inflammatory agents (such as cytokines); and can further augment the efficacy of any antimicrobials co-prescribed. Specific examples of such synergizing agents will be given in a later section.

The practitioner's addition of these or other synergizing agents to augment the oxygenating agent is anticipated by the present invention, and does not change the nature of the novel inventive step. If the practitioner is using any given treatment for microbial diseases (whatever the nature of that treatment), and also adds an oxygenating agent to the mix, he or she is thereby practicing the present invention.

The present invention uses oxygenating agents already known in the art, and takes the novel step, not previously described in the art, of applying said agents to the treatment of infectious disease.

### DESCRIPTION OF PREFERRED EMBODIMENTS

We see from the above discussions that whether an infection is located in ischemic/hypoxic sites, or even in normally oxygenated sites, in either case, increasing the pO₂ levels in said sites can (i) increase the efficacy of antimicrobial agents, and/or (ii) enhance the host's own defenses against the microbes that have invaded the site. The present invention raises the pO₂ in the tissues by delivering an oxygenating agent to the site, such that said increase in pO₂ can be achieved without having to resort to the risks and costs of hyperbaric chambers.

The oxygenating agents, any of which, when administered in the appropriate formulation and at the appropriate site, can be used to practice the present invention are broken down into the following categories (A) oxygen-carrying agents, and (B) entrapped oxygen-generating agents.

A. The oxygen-carrying agents known in the art include, but are not limited to: (i) modifications of naturally-occurring hemoglobins and of heme moieties; (ii) synthetic hemoglobins and hemes; (iii) perfluorocarbons; (iv) aqueous oxygen; and (v) any other type of substance that can dissolve or loosely bond oxygen, and then transport it while in the bloodstream to, and eventually release it into one or more sites, including the target site in need thereof. Detailed descriptions of some of these substances are given later.

B. The entrapped oxygen-generating agents known in the art are those that, (i) when entrapped in appropriate formulations (such as conventional or modified liposomes), and (ii) have subsequently become unentrapped at the site of infection, can then undergo chemical alterations that liberate free oxygen at the site of infection. These include, but are not limited to, entrapped formulations of: (i) hydrogen peroxide, (ii) tetrachlorodecaoxide, (iii) potassium permanganate, and (iv) ozone, all of which react (directly, or with the assistance of certain reagents) with tissues by releasing molecular oxygen (O₂). Note that the present invention does not make claims for the use of use of these substances when in the naked, unentrapped state. This is because, as an example, the prior art teaches that hydrogen peroxide (by itself, unentrapped) can aid in the reduction of bacteria in infected sites such as infected wounds and infected gums

However, the oxygen liberated by these substances when unentrapped cannot diffuse beyond the first few layers of the epithelium or connective tissue on which it has been placed. For ekample, it is known that hydrogen peroxide is used to help oxygenate and debride only the surface area of an infected site. In contrast, the entrapped oxygen-generating agents (as well as the oxygen-carrying agents) of the present invention, when formulated with certain vehicles (as the need may arise, can (i) when administered topically, penetrate through many layers of tissues, and (ii) when injected parenterally, be perfused widely through the circulatory system. These agents of the present invention thereby increase the pO₂ of (i) the intradermal, subcutaneous or dermal tissues, as well as the intramuscular or submucosal tissues; (ii) the parenchymal tissues of an internal organ that they have reached via the circulatory system, and (iii) the hollow interiors of various organs and structures, to which the free oxygen carried by or generated by these agents has diffused) after reaching the vicinity of such hollow organs and structures via the circulatory system. Examples would include, but not be limited to the lumen of the intestines, the interior of the gallbladder, and the sinuses of the skull. Thus, as mentioned, while the unentrapped agents are restricted to topical use, nevertheless, the same agents when in an entrapped formulation and adapted according to the methods of the present invention, can be used to penetrate surface barriers and can also be injected parenterally. Examples of some of the entrapped agents are given later.

Oxygen-Carrying Agents:

The oxygen-carrying agents known in the art (and summarized in Category “A” above) can be broken down into several groups. These groups serve as examples rather than an exhaustive list, other examples (known, or discovered in the future) being apparent to the skilled observer as falling under the present invention:

(1) Those oxygen-carrying agents in which free molecular oxygen (O₂) is transiently physico-chemically bound to a moiety of the agent. Examples include, but are not limited to:
(a) blood substitutes based on cell-free hemoglobin and/or heme products;
(b) encapsulated hemoglobin and encapsulated heme products;
(c) synthetic heme compounds, such as a modified heme compound wherein an alkaneimidazole group binds iron at the proximal sixth coordination site and with four long-chain alkanephosphocholine groups to provide lipophilicity and an oxygen pocket
(d) liposome-encapsulated hemoglobin preparations, which can function as artificial red blood cells; and
(e) modified hemoglobins including, but not limited to, Pyridoxalated Hemoglobin Polyoxyethylene Conjugate (“PHP”); PEG-hemoglobin; o-Raffinose Ploy Hemoglobin (“Hemolink”); Polynitroxyl-Hemoglobin (“PNH”); polymerized human hemoglobin (“Poly SFH—P”); polymerized purified bovine hemoglobin; and cross-linked hemoglobins such as Diaspirin Crosslinked Hemoglobin (“DCLHb”, HEMASSIST™).

(2) Those oxygen-carrying agents, generally in a fluid state, in which oxygen is dissolved but not chemically bound. Examples would include, but are not limited to:
(a) Aqueous oxygen (“AO”), the descriptive name of a recent invention called “TherOxφ” from Wayne State University wherein water is supersaturated with oxygen at a mixture of 1-3 ml of oxygen per gram of water, in a device that delivers the AO by laminar flow into narrow tubing without producing bubble nucleation (despite the high pressure of 100 atmospheres of O₂ (˜1500 psi), pressures previously achievable only with HBO). The AO can then be infused into an artery to produce regional hyperoxemia. The inventors of TherOx®, speaking at an IBC conference on Blood Substitutes (Cambridge, Mass., Nov. 20, 1997), presented data on the use of their invention for hypoxic/ischemic conditions, examples being angioplasty and the treatment of myocardial ischemia.
(b) Another class of oxygen-carrying substances consists of various synthetic chemical compounds, such as the perfluorocarbons (the latter typically being composed of a certain number and permutation of carbon and fluorine atoms). Perfluorocarbons (“PFCs”) are substances of small particle size and low viscosity that are chemically inert in biological systems, and have a high oxygen-carrying capacity relative to plasma and whole blood. Examples of PFCs include, but are not limited to, perfluorodecalin (C₁₀F₁₈), perfluoro-tri-n-proplyamine (C₉F₂₁N), fluoromethylo-adamantane (“FMA”), OXYGENT® (perfluoroctylbromide), PERFLUBRON® (C₈F₁₇Br), and FLUOSOL-DA®. The latter has been approved by the FDA for adjunctive use during coronary angioplasty, where it is infused through the lumen of the catheter to provide oxygen to arterial segments distal to the inflated balloon, the product having been demonstrated to decrease the myocardial ischeinia associated with angioplasty. Most of the compounds listed above are described in, Blood Substitutes: New Challenges, ed. by R. M. Winslow, K. D Vandegriff and M. Intaglietta, Boston: Birkhause Publishers, 1996; and also in Scientific Basis of Transfusion Medicine: Implications for Clinical Practice, ed. by K. Anderson and P. Ness, Philadelphia: W. B. Saunders Company, 1994. Perfluorodecalin and perfluorotri-n-propylamine are briefly described in USP DI “Approved Drug Products and Legal Requirements”, 17th Edition, 1997.] The two agents are formulated together in a product listed therein as “perfluorochemical emulsion”, the description of which is given as follows: “Stable emulsion of synthetic perfluoro-chemicals . . . in Water for Injection. Also contains Polaxemer 188 (a nonionic surfactant which is a polyolyethylene [160]-polyoxypropylene [30] block copolymer), glycerin, egg yolk phospholipids (a mixture of naturally occurring phospholipids isolated from egg yolk), dextrose (a naturally occurring sugar), and the potassium salt of oleic acid (a naturally occurring fatty acid), plus electrolytes in physiologic concentrations”.

Additional information about certain perfluorocarbons was presented on May 15, 1997 by the cosmetics company Dragoco at the International Business Conference which was entitled “Delivery Technologies for Cosmetic Ingredients”. Dragoco's handouts state: (1) “Perfluorated carbon compounds are substances suitable for use as blood replacements, being capable of dissolving large quantities of oxygen”; (2) “When stabilized with physiological emulsifiers, such nanoemulsions can transport oxygen and deliver it to the organism”; (3) “Unfortunately, such nanoparticles show little or no ability to penetrate the barrier of the skin”.

For that reason, Dragoco incorporated the perfluorocarbon nanoemulsion into a liposome. Their handout presents data showing that after 14 days of twice-daily topical administration, this liposomal perfluorocarbon formulation had increased the pO₂ in the skin of aging human volunteers by approximately 100%.

For example, their handout goes on to report that the 14 days of treatment had produced “a 10% decrease in the number of wrinkles, 40% decrease in the depth of wrinkles, 30% increase in skin moisture content, and 10% increase in skin thickness. . . . Because of the energy provided by the oxygen we deliver, poorly supplied skin recovers the ability to regenerate the outermost layers of the skin”. Dragoco was focused on the potential of such an oxygen-carrying system as a method to overcome the effects of ischemia (and ischemia alone). They are silent on its use in treating bacterial infections (whether in ischemic or normal tissues), the subject of infections not being relevant to their goals and purposes. Therefore, they do not teach the use of oxygenating substances to treat infectious disease.

The journal articles cited by Dragoco in the handout, relevant, as said, to the use of this oxygenating agent against ischemia, are: (1) Stanzl et al. “A new cosmetic product containing molecular oxygen” Euro Cosmetics, 1/93, p. 39. (2) Stanzl et al. “The effectiveness of molecular oxygen in cosmetic formulations” Intl. J. of Cosmetic Sci., 18:137-150, 1996.

PFCs are preferred over the heme- and hemoglobin-based oxygenating agents for the purposes of the present invention, because the iron and/or the heme in the non-preferred compounds are toxic to macrophages and to cells of the endothelial lining. In addition, there are reports that hemoglobin-based products might lead to an increased risk of infections, perhaps due to the participation of hemoglobin in the specific binding of bacterial endotoxins. Given that the oxygenating substances as used in the present invention would be administered specifically to people who have already contracted (or are at risk of contracting) an infection, it may be desirable to avoid the adverse effects described above if at all possible. However, whichever oxygen-carrying substances may prove over time to be most applicable, the use of any of them in an effective dosage, so as to attain the desired result in the treatment and/or prevention of microbial infections, would constitute the practice of the present invention.

Entrapped Oxyyen-Generating Agents:

The entrapped oxygen-generating agents known in the art (and summarized in Category “B” above) can be broken down into several groups (which are meant to serve as examples and not an exhaustive list; other examples, known or later to be discovered would be apparent to the skilled observer as falling under the present invention):

(1) In the case of (as examples) hydrogen peroxide (H₂O₂), tetrachlorodecaoxide, and ozone (O₃): Entrapment of the oxygen-generating substance in pH-sensitive vehicles known in the art, such as globular-, cochlear- or dendrimer-shaped materials such as lipids (liposomes), amino acids, polymers, or any other suitable formulation now known or later known in the art. When the pH-sensitive vehicle. degrades at the sites of infection (which sites are generally acidic), the oxygen-generating substance is released into the exterior milieu, where the simple interaction with the tissues causes it to decompose, generating free molecular oxygen.

(2) In the case of (as an example) potassium permanganate (KMnO₂): Entrapment in the inner compartment of pH-sensitive vehicles known in the art (such as multilammelar liposomes), of the oxygen-generating substance; and the entrapment, in the outer compartment of said vehicle, of a reducing substance; such that the degradation of the pH-sensitive vehicle at the sites of infection (which sites are generally acidic) releases, into the exterior milieu, both the oxygenating agent and the reagent that will reduce it, thereby releasing free molecular oxygen.

Examples of Sites at Which or to Which the Oxylenating Agents can be Administered.

While oxygenating agents can harm bacteria and/or assist host defenses at any site, they are most critically needed when the infected tissue is poorly-oxygenated. For purposes of clarity, poorly-oxygenated tissues, and the infections typically found therein, are described as follows:

(1) Those tissues that may once have been well-vascularized, but which have subsequently become poorly vascularized, due to (a) the normal processes of aging (where the blood flow to the skin progressively diminishes, leading to ischemic skin ulcers that can then become infected); (b) disease processes (such as pressure sores that occur in the context of small vessel disease in diabetics, which sores then become infected); and (c) physical trauma (such as burns that subsequently become infected). In the early stages of pressure sores, and of the related condition cellulitis, it would be important to administer the oxygenating agents of the present invention as early as possible in the disease process, in order not only to keep bacteria in check at these sites, but also to utilize the known ischemia-reversing abilities of these agents so as to prevent further tissue necrosis. It is axiomatic that there is vicious cycle wherein (a) the greater the degree of tissue necrosis, the-greater the ability of bacteria to colonize the developing lesion and to penetrate more deeply therein (because barriers to such invasion break down), and (b) the greater the degree of said bacterial colonization and penetration, the more the process of tissue necrosis is hastened.

(2) Those tissues that were inherently never well-vascularized, and which are therefore chronically subject to low oxygen tensions, such as would obtain in (a) infections of bones (e.g. osteomyelitis), joints, eyes (e.g. cytomegalus virus), middle ear, and sinuses; (b) infections of the superficial layers of the skin (e.g. acne and impetigo); (c) infections of the male and female genitourinary organs, such as syphilis, gonorrhea, chlamydia, ovosalpingitis, and acute or chronic infections of the kidneys (pyelonephritis), the ureters, the urinary bladder, the urethra, the prostate, or the epididymis; (d) infections of the mucous membranes generally, examples being (i) the linings of the upper and lower respiratory tracts (as in bacterial and viral pneumonias), (ii) the linings of the upper and lower gastrointestinal tract as in Crohn's disease, ulcerative colitis, and gastric and duodenal ulcers (that may be infected with Helicobacter pylori); and (iii) infections of the oral cavity (e.g. periodontitis).

(3) Abscesses, whether small and superficial (such as boils and furuncles), or deep (such as peritonitis, empyema, perineal abscesses, or other infected body cavities/tissues).

(4) Infections in the lumen of hollow organs, and infections in the tubes afferent or efferent to such organs. Examples would include but not be limited to infections of the gallbladder or of the common duct, e.g. a bacterial infection (such as with E. coli) or a parasitic infection (such as with Giardia lamblia that have migrated to the gallbladder).

(5) Intracellular locations, such as a lymph node where white blood cells are infected with a bacteria (e.g. Mycobacterium tuberculosis) or with a virus (such as the Human Immunodeficiency Virus).

While it is in poorly-oxygenated tissues such as the above that the use of the present invention may be most critically needed, nevertheless the present invention is not limited to such tissues, for it may also useful under normal oxygen tensions, for example where (i) the infecting microbe happens to be susceptible to being harmed by higher-than-normal oxygen tensions, and/or (ii) the tissue site is undergoing breakdown (e.g. in the case of early-stage pressure sores resulting from bed rest). In such instances, the improved oxygenation of the present invention would tend to lessen the rate of such tissue breakdown, and, as a result, the risk of infection therein would be reduced. While examples of microbes that can be damaged by oxygen would logically include anaerobic and microaerophilic bacteria, nevertheless even certain aerobic bacteria can be damaged by an increased pO₂. Thus, for example, HBO has been used to enhance the efficacy of antibiotics in treating infections with aerobic as well as anaerobic bacteria. In the present invention, oxygenating agents are used in the place of HBO.

Additional examples of infections where an increase in pO₂ might be helpful, even though the infection is located in a well-oxygenated tissue, would include: hepatitis A, B or C infections, where the infecting agent in question is residing inside parenchymal cells of the liver; and HIV, where the infecting agent resides in T cells located not only in the lymph nodes, but also in the circulatory system. In many such infections, there is evidence that an increase in pO₂ can improve the killing dynamics of the host cells (such as their ability to generate free radicals, and the efficacy of cytokines acting therein).

Examples of Formulations and Delivery Systems Appropriate for Use at the Various Sites.

Where the infection is relatively superficial, the oxygenating agent can be administered topically for intradermal penetration, by locally applying any appropriate formulation of an oxygenating agent (with or without an appropriate antimicrobial agent).

In those cases where the oxygenating agent in question does not readily penetrate the superficial layers of the epithelium, and where the infection is either (i) in the deeper regions of the dermis/subderrnis or (ii) is not accessible at all to topical administration, a variety of pharmaceutical vehicles and modes of administration can be employed to effect penetration. The degree and rate of penetration of the vehicles are expected to be increased in tissues that are infected (as compared to tissues that are not infected), due to the acidic and edematous conditions caused by infection and inflammation, along with the general increase in permeability of connective tissue and blood vessels that is concomitant with those conditions. Such vehicles and/or modes of administration would include, but are not limited, to:

A. Transdermal patches, many of which are known to the skilled artisan.

B. Encapsulated and micro-encapsulated formulations. Numerous encapsulation technologies are known to the skilled artisan. [See e.g. Encapsulation and Controlled Release, Ed. by D. R. Karsa and R. A. Stephenson, Publ. by Royal Society of Chemistry, Cambridge, 1993.] For example, the skilled artisan would be conversant with the use of (i) liposomes, (ii) non-phospholipid liposome-type formulations, (iii) dendrimers, (iv) cochlear-shaped lipid materials, and (v) micro-encapsulated materials, all of the above being known in the art, and being able, with appropriate modifications, to entrap the oxygenating agent until there has been degradation of said vehicle, with subsequent release of the oxygenating agent into the deeper layers of the integument targeted.

C. Emulsions and gels, known to the skilled artisan that, while not encapsulating in the strict sense of the term, might nevertheless have properties that prevent most of the oxygen from being liberated until sufficient penetration of the dermal layers has been achieved.

D. Bandages and dressings, known to the skilled artisan, that are to be placed onto the surface of wounds and incisions, and wherein the oxygenating agent is interspersed via microencapsulation or other technologies suitable to liberate the oxygen over time. The compositions of the underlying bandages and dressings that are suitable for such purposes are known to the skilled artisan, and would include (but would not be limited to): polyurethane and other polymer thin films; hydrocolloids and hydrogels; calcium alginates; and collagen-based composites. The bandages and dressings can contain any number of other reagents known in the art that promote wound healing and/or antisepsis, the inventive step herein being the addition of an oxygenating agent E. Packing materials (such as Iodoform® gauze) that are inserted into wounds and incisions to promote sterilization, drainage, and healing, and wherein the oxygenating agent is interspersed via microencapsulation or other technologies suitable to liberate the oxygen over time. The compositions of the underlying packing materials that are suitable for such purposes are known to the skilled artisan, and would include (but would not be limited to): polyurethane and other polymer thin films; hydrocolloids and hydrogels; calcium alginates; and collagen-based composites. The packing materials can contain any number of other reagents known in the art that promote wound healing and/or antisepsis, the inventive step herein being the addition of an oxygenating agent.

E. In the case of periodontal infections, the oxygenating agent can be applied: (i) as a toothpaste, gel or other suitable formulation for the patient's own use for penetrating the oxygenating agent into the gums, and/or (ii) as a packing material that a dentist can insert into the gingival pockets (similar in many respects to the antibiotic-releasing forrmulations dentists currently use as a packing material in the gingival space). The formulations of.the toothpastes, gels and packing materials are known in the art, the inventive step herein being the addition of an oxygenating agent in a suitable formnulation.

F. Aerosols, e.g. for sprays that reach the nasal passages and/or the inuses, and for inhalation delivery to the lungs. There are many spray and inhalation formulations known in the art, any one of which can be used in the present invention, the inventive step herein being the addition of an oxygenating agent. An example of such an aerosol is the type represented by the PROVENTIL™ inhaler manufactured by Schering-Plough, the propellant of which contains oleic acid, trichloromonofluoromethane, and dichlorodifluoromethane. The concentrations of the propellant ingredients and emulsifiers are adjusted if necessary based on the oxygenating agent being used in the treatment.

G. By direct injection or instillation, in those cases where the infected tissue consists of a deep area not accessible to topical therapies. Examples would include but not be limited to: ocular infections (where the agent is directly injected), abscesses of the body cavities (where, again, the agent is directly injected), and bone infections with fistulae (where the agent is instilled into the fistula).

H. All traditional parenteral routes of drug administration would be applicable, such as injection by the following routes: subcutaneous, intramuscular, intravenous, intra-arterial, intraperitoneal, intracardiac, intrapericardiac, by lumbar puncture, intrathecal, and by burr hole for direct instillation into the meninges or into the parenchyma of the brain itself (in the case of an abscess).

I. The oxygenating agents can be administered to the various internal mucosal surfaces. Thus, for example, the agents can be administered: per os (in a mouthwash formulation or gel application); per vagina or per rectum, in suppository or enema formulations; and by endoscopy, for example in infections of the epiglottis, the bronchi, the lungs, the stomach (or duodenum), the uterus (or fallopian tubes), and the upper, middle or lower segments of the urinary tract. In the more distal regions of these mucosal surfaces, meaning those that are closest to the orifice thereof, topical formulations similar or identical to those described above for the skin can be employed, wherein the liposomes or other vehicles of said formulation can enable the oxygenating agent or its oxygen load to penetrate deeply into the submucosal regions.

In all the examples cited above, the excipients which can be used as a vehicle for the delivery of the oxygenating agents will be apparent to those skilled in the art. For example, the oxygenating agents can be in lyophilized form and can then be dissolved in water or saline just prior to administration by injection. Diluents and stabilizers known to the skilled artisan can be added, if and as necessary.

The oxygenating agent and an appropriate antimicrobial agent can be co-administered in the same vehicle (e.g., by co-encapsulation) or in the same injection or IV drip, but it is not necessary for the practice of the invention that the two types of agents be co-administered. In fact, the oxygenating agent and the antimicrobial agent can be administered by different routes and at different times (for example, where the antibiotic is administered topically and the oxygenating agent by injection, or vice versa).

It is contemplated that a variety of inmune modulators and other agents can be administered with the oxygenating agents, whether co-formulated or administered separately. The modulators would include but not be limited to:

(a) Antioxidants, such as superoxide dysmutase (“SOD”), vitamin E (tocopherol), catalase, and ascorbic acid.

(b) Growth factors, such as but not limited to the various epithelial growth factors (EGFs), interferons, cytokines, chemokines, and MHC Type II-inducing or—modulating factors:

(c) Endotoxin antagonists, such as steroids, monoclonal antibodies, or reconstituted HDL.

These and other synergizing agents can be co-administered with the oxygenating agents of the present invention, in order to:

(a) Protect against any free radicals that might (i) be engendered by the respiratory oxidative stress (“ROS”) caused by certain infections (such as the influenza virus), or (ii) that might be engendered by the oxygenating agents themselves.

(b) Promote more rapid healing of wounds.

(c) Counter the actions of various pro-inflammatory agents such as cytokines, and/or

(d) Further augment the efficacy of any antimicrobials co-prescribed.

Examples of Microbes Targeted by the Oxygenating Agents

The present invention does not claim to treat all microbial infections, as there may be some pathogens that are not affected (i) directly by an increase in pO₂, or (ii) indirectly by the improvement brought about when said increase in pO₂ potentiates either the efficacy of antimicrobials or the efficacy of bost antimicrobial defenses. However, the practitioner will be able to predict, or will be able to determnine empirically, which of such microbes are generally susceptible to the direct or indirect effects of an increased pO₂. When the practitioner uses an oxygenating agent (as opposed to HBO therapy) as part or all of the treatment of any infection, he or she is practicing the present invention.

The infections that may be treated by the present invention can be from any microbe, including, but not limited to: bacteria, viruses, yeasts, fungi, rickettsiae and parasites (the latter whether single- or multi-cellular).

While it is contemplated that the present invention can be used to treat any microbial infection in an animal or human, it is particularly contemplated that the methods described herein will be very useful as a therapy in infections caused by drug-resistant microbes, where every advantage is needed to kill the microbe and to support the host defenses. For bacterial targets in particular, experts report that at the present time, the drug-resistant bacterial species and strains listed below (see, for example, Gibbons, Science, 257:1036-1038, 1992) represent the greatest threat to mankind:

1. All of the clinically important members of the family Enterobacteriaceae, most notably, but not limited to, the following:
a) All the clinically important strains of Escherichia, most notably E. coli.
b) All the clinically important strains of Klebsiella, most notably K. pneumoniae.
c) All the clinically important strains of Shigella, most notably S. dysenteriae.
d) All the clinically important strains of Salmonella, including S. abortus-equi, S. typhi, S. tylphimurium, S. newport, S. paratyphi-A. S. paratyphi-B S. potsdam, and S. pollurum.
e) All the clinically important strains of Serratia, most notably S. marcescens.
f) All the clinically important strains of Yersinia, most notably Y. pestis.
g) All the clinically important strains of Enterobacter, most notably E. cloacae.

2. All the clinically important Enterococci, most notably E. faecalis and E. faecium.

3. All the clinically important Haemophilus strains, most notably H. influenzae.

4. All the clinically important Mycobacteria, most notably M. tuberculosis, M. avium-intracellulare, M. bovis, and M. leprae.

5. Neisseria gonorrhoeae and N. meningitidis.

6. All the clinically important Pseudomonads, most notably P. aeuruginosa.

7. All the clinically important Staphylococci, most notably S. aureus and S. enidermidis.

8. All the clinically important Streptococci, most notably S. pneumoniae and S. pyogenes.

9. Vibrio cholerae.

There are additional bacterial pathogens too numerous to mention that, while not currently in a state of antibiotic-resistance crisis, nevertheless make excellent candidates for treatment with oxygenating agents, in accordance with the present invention. Thus, all bacterial infections that are susceptible to increased pO₂ levels or to improved host defenses can be treated using the present invention.

Various other species of microbes (viruses, yeasts, parasites, rickettsiae, etc.) have become multidrug resistant as well. Thus the present invention will be particularly useful as a treatment or co-treatment of such other species of microbes, some but not all of which are listed in the next section.

Examples of antimicrobial Agents that can be Co-Administered with the Oxygenating Agents.

The oxygenating agents of the present invention can be used as a stand-alone therapy, or as an adjunctive therapy for the treatment of any microbial infection that is susceptible to increased pO₂ levels. Numerous antimicrobial agents would be useful in combination with said oxygenating agents for treating such infections. Examples of suitable antimicrobial agents that could be co-administered with the oxygenating agents of the present invention would include, but would not be limited to, the following: (i) antibiotics (meaning the antibacterial chemicals secreted by various bacteria, fungi and other microorganisms); (ii) chemotherapeutic drugs (meaning synthetic antibacterial chemical agents, such as sulfa drugs); (iii) bacteriophages; (iv) bacteriocins; (v) bacteriocin-like inhibitory substances (“BLIS”); (vi) lantibiotics; (vii) members of the “defensins”, a group of naturally-occurring antibacterial substances secreted by the skin, mucous membranes, white blood cells and/or other structures of vertebrates and non-vertebrates, important examples being Bacterial Permeability Increasing Protein (“BPI”) and the “magainins”; (viii) the various antiviral, antifutngal and antiparasitic drugs, whatever their chemical composition or mode of action; and (ix) the various sterilizing/disinfecting agents that are used or can be used topically, in combination with the oxygenating agents of the present invention. However, the present invention is not limited to the classes of antimicrobial agents listed above, as one skilled in the art could easily determine other antimicrobial agents or classes of agents that would be useful in combination with the oxygenating agents of the present invention. The anti-infective agents used to treat such microbes are collectively referred to herein as “antimicrobials”.

The following tables provide examples of some, but not all, of the antimicrobial agents that can be combined with the oxygenating agents of the present invention to increase the efficacy of the antimicrobial(s) in question. In all instances where the microbe cited in the table is a bacterium, the bacterial target specified can in all cases also be killed by phages and/or by bacteriocins, so these latter agents are incorporated by reference. The efficacy of the phages would (like that of the antibiotics) be improved by an increased pO₂, because (i) bacteria are less likely to replicate under lower oxygen tensions, and (ii) phages require the bacterial target to be replicating in order to produce daughter phages that can lyse said bacteria, thus an increase in pO₂ will favor phage replication and, thereby, the bactericidal action of phages.

In the following tables, the left-hand column lists examples (non-inclusive) of various kingdoms and species of microbes, infections from which can be attenuated by the present invention's increase of pO₂. The right-hand column lists examples (non-inclusive) of the corresponding antimicrobial agents (and/or groups of agents) whose efficacy can be enhanced by said oxygenating agents of the present invention.

| | |
|---|---|
| | Antibacterial agents. Note: For all bacterial |
| | targets, it is understood that the bactericidal |
| | action of bacteriophages will be enhanced by an |
| | increased pO₂ (see text), so that it is not |
| I. Bacterial Pathogens | necessary to list the phages for each example. |

E. coli

| | |
|---|---|
| Uncomplicated urinary tract infection | Trimethoprim-sulfamethoxazole |
| | (abbrev.TMO-SMO), or ampicillin; 1st |
| | generation cephalosporins, ciprofloxacin, |
| | levoquin, noroxin, floxin, furadantin. |
| systemic infection | Ampicillin, or a 3rd generation |
| | cephalosporin; aminoglycosides, |
| | aztreonam, or a penicillin + |
| | a pencillinase inhibitor |
| Klebsiella pneumoniae | 1st generation cephalosporins; 3rd |
| | generation cephalosporins; cefotaxime, |
| | moxalactam; amikacin, quinolones such as |
| | ciprofloxacin, levoquin, combination |
| | antibiotics; zosyn |
| Shigella (various) | Ciprofloxacin; TMO-SMO, ampicillin, |
| | chloramphenicol |
| Salmonella: | |
| S. typhi | Ciprofloxacin, chloramphenicol; ampicillin |
| | or TMO-SMO |
| non-typhi species | Ampicillin; chloramphenicol, TMO-SMO, |
| | ciprofloxacin |
| Yersinia pestis | Streptomycin; tetracycline, ciprofloxacin, |
| | chloramphenicol |
| Enterobacter cloacae | 3rd generation cephalosporins, gentamicin, |
| | or tobramycin; carbenicillin, amikacin, |
| | aztreonam, imipenem |
| Haemophilus influenzae: | |
| meningitis | 3rd generation cephalosporins; ampicillin, |
| and | chloramphenicol |
| other H. influenzae Infections | ampicillin; TMO-SMO, cefaclor, |
| | cefuroxime, ciprofloxacin |
| Mycobacterium tuberculosis and | isoniazid (INH) + rifampin or rifabutin, the |
| M. avium-intracellulare | above given along with pyrazinamide +/ |
| | or ethambutol |
| Neisseria: | |
| N. meningitidis | Ampicillin, unasyn, penicillin G; |
| | chloramphenicol, or a sulfonamide |
| N. gonorrhoeae: | |
| penicillin-sensitive | penicillin G; spectinomycin, ceftriaxone; |
| penicillin-resistant | spectinomycin, cefuroxime or cefoxitin, |
| | ciprofloxacin |
| Pseudomonas aeruginosa | Tobramycin or gentamycin |
| | (+/− carbenicillin); amikacin, ceftazidime, |
| | aztreonam, imipenem |
| Staphylococcus aureus | |
| non-penicillinase-producing | Penicillinase + penicillin G; 1st generation |
| | cephalosporins, vancomycin, imipenem, |
| | erythromycin |
| penicillinase producing | a penicillinase-resisting penicillin; 1st |
| | generation cephalosporins, vancomycin, |
| | imipenem, erythromycin |
| Streptococcus pneumoniae | penicillin G; 1st generation cephalosporins, |
| | erythromycin, chloramphenicol |
| Vibrio cholerae | tetracycline; TMO-SMO |

| II. Viral Pathogens, including | Antiviral Agents/Groups, including |
|---|---|
| but not limited to: | but not limited to: |
| HIV | AZT, ddI, ddC, d4T, 3TC, neveripine, |
| | delavirdine, saquinivir, crixovan, |
| | ritonavir, viracept, protease |
| | inhibitors |
| Herpes simplex virus | Zovirax; famvir, valtrex |
| Hepatitis virus (A, B, C, D, E, | Interferons; alpha interferon, lubucavir, |
| and any additional viruses that | 3TC |
| may in the future be discovered) | |
| Varicella-Zoster Virus | Famvir, valtrex |
| Influenza Virus | Amantadine |
| Respiratory Syncytial Virus | Ribavirin |

| IV. Fungi, including but | Antifungal Agents/Groups, |
|---|---|
| not limited to: | including but not limited to: |
| Mucoraceae | Amphotericin B, abelcet |
| Histoplasma | Amphotericin B, abelcet, |
| Blastomyces | Amphotericin B and abelcet |
| Coccidioides | Amphotericin B, abelcet |
| Aspergillus | Amphotericin B or abelcet |
| Sporotrichosis | Potassium iodide, amphotericin B, abelcet |
| Dermatophytes | Lamusil, ketoconazole, itraconazole |
| Trichosporin | Amphotericin B, abelcet |
| Allescheria boydii | Amphotericin B |

| V. Parasites, including but | Antiparasitic Agents/Groups, |
|---|---|
| not limited to: | including but not limited to: |
| Giardia lamblia | Flagyl |
| Entamoeba histolytica | Flagyl |
| Entamoeba histolytica | Iodoquin |
| Dientamoeba fragilis | Iodoquin |
| Balantidium coli | Flagyl |
| Naegleria | Amphotericin B |
| Acanthamoeba | Flagyl |
| Trypanosome spp. | Eflornithine, melarsoprol B |
| Leishmania spp. | Ketoconazole, amphotericin B, |
| | stibogluconate |
| Toxoplasma gondii | Sulfonamides |
| Pneumocystis carinii | Bactrim, pentamidine, atovoquine, |
| | trimetrexate |
| Plasmodia falciparum, etc. | Primaquine, mefloquine, atabrine, quinine, |
| | etc. |
| Schistosomiasis | Praziquantel |
| Ascaris | Mebendazole, albendazole |
| Hookworm | Mebendazole |
| Trichuris | Piperazine |

| Rickettsiae, including but | Antirickettsial Drugs/Agents, |
|---|---|
| not limited to: | including but not limited to: |
| Rickettsiaceae: ricketsii, | tetracycline, ciprofloxacin |
| akari, prowazekii, typhi, | |
| tsutsugamushi | |
| Rochalimeae: quintana | tetracycline, Biaxin, zithromax, floxin, |
| Coxiella: burnetii | levoquin |
| Ehrlichia: sennetsu, canis, | erythromycin, ciprofloxacin, tetracycline |
| equi, phagocytophila, risticii | |
| Bartonella: bacilliformis | erythromycin, tetracycline, ciprofloxacin |
| Chlamydia: trachomatis, | Zithromax, Biaxin, tetracycline, |
| psittaci | erythromycin |
| Mycoplasma | Zithromax, ciprofloxacin, tetracycline |

The dosage of the antimicrobial component of the combined preparation is contemplated to be equal to or less than the dosage of such agents when used alone. Such dosages are administered in conjunction with the oxygenating agents until complete elimination of the microbe is achieved, or until their numbers have been reduced to the point where the host defenses, no longer being overwhelmed, can kill any remaining bacteria.

Another embodiment of the present invention is the development of methods to treat bacterial infections in animals and humans, through therapy using the oxygenating agents (with or without antimicrobial agents or other synergizing agents). The present invention is not limited to (i) a specific oxygenating agent, (ii) a specific microbial infection in need of treatment, nor (iii) to a specific antimicrobial agent. Rather, the present invention can be utilized to treat any and all infections in humans and other animals, where either (i) the microbes causing said infections are susceptible to the increase in pO₂ or (ii) the host defenses against the microbes can be significantly enhanced by said increase in pO₂.

Intended Recipients of the Present Invention

The animals to be treated by the methods of the present invention include, but are not limited to: man, his domestic pets, livestock (including poultry and cattle), aquaculture, and the animals in zoos and in aquatic parks (such as whales and dolphins).

All books, articles and patents cited in this specification are incorporated herein by reference in their entirety.

The following examples are illustrative of the present invention; however, the practice of the invention is not limited or restricted in any way by them.

### EXAMPLES

### Example 1

### Infected Ischemic Wound: Use of a Topical Oxygenating Agent for Penetration Into the Intradermal and Subcutaneous Spaces

Step 1. Establishing the Infection:

A diabetic mouse model of infected partial-thickness burn wounds is used, by modifying the design using non-diabetic mice developed by Cribbs, et al. A Standardized Model of Partial Thickness Scald Burns in Mice. Journal of Surgical Research. 80: 69-74, 1998. In this model, a partial-thickness scald wound is created, as verified by histological specimens, by exposing the dorsum of anesthetized obese diabetic mice to 60° C. water for the requisite number of seconds. The burned areas are then inoculated with 5×10ˆ5 cfu of a strain of Pseudomonas aeruginosa that is non-virulent, to obtain a chronic, nonlethal wound. On the fifth day following burning, the eschars (if any) are excised from the wound, and the wounds are then observed clinically and histologically for the degree of healing and the bacterial counts.

Step 2. Treatment Modalities: The rats are Broken Out Into Four Groups:

Group 1. Perfluorocarbon alone is topically applied twice daily, for 15 days, encapsulated in a liposomal formulation containing approximately 1 mL of the perfluocarbon perfluorodecalin per liposome (as Coty, Inc.'s product called A*O*C*S*®).

Group 2. Antibiotic alone is topically applied twice daily, for 15 days, in the form of one gram of a topical formulation of the antibiotic Cleocin T gel 1%.

Group 3. Combined antibiotic and A*O*C*S*®, applied topically at the same time, as per above.

Group 4. Placebo is topically applied twice daily, for 15 days, consisting of (a) liposomes containing normal saline, and of (b) the base vehicle in which the antibiotic was formulated, which is essentially allantoin and various excipients.

Directions:

In all cases, one gram of the liposomal preparation (or the placebo control) and one gram of the antibiotic preparation (or its placebo control) are applied to the site of the infected ischemic skin twice daily, approximately 6 hours apart, for 14 days. On each occasion the lesion is covered afresh with sterile gauze, lined on the. skin side with an impermeable layer known to not absorb the liposomal formulation or the antibiotic formulation. The bandage is secured in such a manner that the animal cannot pull or chew it off, and therefore cannot lick off the medication/placebo.

Step, 3. Preparation of Specimens for Analysis:

One animal from each group is sacrificed. Every 4^{th} day during the course of the 14 day treatrnent, one animal from each group has its ischemic skin lesion biopsied under aseptic conditions; this material is then weighed, and diluted 1:10 in pH 6.0 PBS to test for the number of bacterial colonies per gram of skin structure (see procedure below). On day 15, all animals are sacrificed humanely by IM injection of standard euthanizing agents. To examine the lesions histologically at the time of sacrifice, for the degree of healing or lack thereof, as well as for the number of white blood cells per field (a sign of infection and inflammation), the ischemic skin area is removed surgically. and is divided by scalpel cuts into four rectangles roughly equal in area, designated sections A, B, C and D, where sections A and D are the rectangles on the periphery (left and right sides) of the lesion, and sections B and C are the rectangles in the middle of the lesion.

Sections A and C (one from the periphery and one from the center) are weighed, and then gently macerated without heating, the macerate then being suspended in 0.5 cc of normal sterile saline, which is then poured onto a petri dish containing cetrimide for the selective isolation and presumptive identification of P. aeruginosa. The petri dish is then incubated for 48 hours at 37 degrees centigrade.

Sections B and D (again, one from the periphery and one from the center) are weighed, and then cut by vertical scalpel slices into smaller strips approximately ⅛ inch wide, which are mounted histologically for observation under a light microscope. The sections are then graded by an expert blinded for the conditions of the experiment, who scores each on a scale ranging from complete necrosis to complete healing (measured as % of normal thickness of the epidermis, among other variables).

Step 4. Results:

Bacterial Counts:

From each of the four experimental arms, counts are made of the cfu of the bacteria grown from the macerated skin suspensions that had been spread on the petri dish.

Histology:

From each of the four experimental arms, measurements are taken for skin thickness (representing healing of the lesion) and for the approximate number of inflammatory cells (PMNs, etc.) per cubic millimeter of skin necropsied.

The results indicate that the combination of oxygenating agent and antibiotic is more efficacious in reducing bacterial counts than the antibiotic alone. The combination also gives an increase in the percentage of healing and a greater decrease in the percentage of inflammatory cells infiltrating per cc of skin.

### Example 2

### Injection of an Oxygenating Agent Into an Ischemic Subcutaneous Infection

Procedures outlined by Onderdonk's group (see e.g. (1) Onderdonk, A. B. et. al., “Experimental Animal Models for Anaerobic Infections”. Reviews of Infectious Disease, Vol. 1, No.2, March-April 1979, and (2) Joiner, K. A. et. al., A Quantitative Model for Subcutaneous Abscess Formation in Mice, Br. J. Exp. Path. (1980) 61, 97-107) are modified so that the subcutaneous access is created on the leg (instead of on the flank, as described by Onderdonk).

Step 1. Establishing the Infection:

The inoculum consists of (a) colonies of Bacteroides fragilis and Staphylococcus aureus each of which been adjusted to 3×10⁸ CFU/ml by adding sterile peptone-yeast-glucose (PYG) that has been prereduced; and (b) an adjuvant consisting of autoclaved mouse caecal contents in PYG. 0.25 ml of the inoculum is injected s.c. into the shaved and depilated left flank of mice, in the manner described by Joiner et. al. (which includes tracking the needle as the material is injected).

Step 2. Timing of Treatment

The animals are divided into two groups, in terms of timing:

(a) Group 1: Receives the treatment modalities described below, starting when the following objective signs of pre-abscess inflammation are observed (generally around 48-72 hours): the margins are indistinct and generally compressible.

(b) Group 2: Receives the treatment modalities described below, starting when the following objective sign of a maturing abscess is observed (generally around 72 hours): a well-delineated s.c. nodule is readily visible and palpable, but not yet firm.

Step 3. Treatment Modalities:

The mice in each of the timing groups are assigned to one of four treatment arms, wherein, for 15 consecutive days starting from the commencement of treatment dictated above, each animal will receive two injections per day (8 hours apart) of one or the other of the materials described below. The material is injected directly into the area of inflammation or abscess, as the case may be, and the needle is tracked during the course of injection as described in Joiner. The materials to be injected are: (a) 1.0 ml of a solution of an oxygenating agent (in this case PERFLUBRON®; (b) 1.0 ml of an antibiotic (in this case clindamycin, in a solution containing 150 mg/ml of the drug; (c) more or less simultaneous injection of both PERFLUBRON® and clindamycin (in the same concentrations and amounts as described above, but administered in separate syringes), or (d) 1.0 ml of sterile normal saline.

Step 4. Evaluation and Quantitation of Results:

The animals are assessed daily, using calipers to measure the size of the developing abscess, where the product of the longest diameter (D) and corresponding perpendicular diameter (d) are recorded as “external area” (Dxd). Each animal is sacrificed on the twentieth day after bacterial inoculation, using 100% CO₂. Within 5 min of sacrifice, the abscesses are removed by wide dissection and are processed in two ways:

(a) For histological examination: the abscesses are immediately placed in 20 ml of 10% formalin and processed for quantitation of abscess size as per the guidelines in Joiner et. al.

(b) For quantitative bacterial counts in the purulent exudate: the abscesses are incised by aseptic techniques. An aliquot of 0.1 ml of purulent material is removed, added to 9.9 ml of prereduced VPI dilution salts, and transferred inmuediately to an anaerobic chamber. The specimen is homogenized with a tissue grinder, serial 100-fold dilutions are made, and 0.1 ml of each dilution is plated on prereduced brucella blood base agar. Colonies are counted after incubation for 48 hours, and results are expressed as CFU/ml pus.

Step 5. Results:

The experimental results reveal that the combination of oxygenating agent and antibiotic is more effective in reducing the bacterial counts than the antibiotic alone.

### Example 3

### Intra-Arterial Infusion of an Oxygenating Agent (Aqueous Oxygen, “AO”) to Produce Regional Hyperoxemia for Curing an Ischemic Subcutaneous Skin Infection

Step 1. Establishing the Infection:

A rabbit model of infected ischemic subcutaneous ulcer is established according to the method of Joiner, K. A. et. al. “A quantitative model for subcutaneous abscess formation in mice”, Br. J. Exp. Path. (1980) 61, 97-107. The procedures are modified in that the infection is induced in the subcutaneous area of the thigh instead of in the flank.

The inoculum consists of a subcutaneous injection of 10⁹ cfu of Bacteroides fragilis per ml, injected into the left thigh, in each of 16 animals.

Aqueous Oxygen is a highly O₂-saturated bubbleless infusate containing 1-2 ml O₂ per gram. In all cases where AO is infused, the method of administration is as follows: a catheter is inserted into the femoral artery on the side contralateral to the infection and is threaded in the direction of the heart until there is radiographic confirmation (using contrast medium) that the tip of the catheter is in the distal aorta (i.e., just caudal to the renal arteries). The AO is then infused, so that the blood carrying the AO reaches the left and right femoral arteries, and, therefore, the lesion in the left thigh.

Step 2. Treatment Modalities:

The rabbits are assigned to one of four groups, and treated twice each day for 15 days.

Group 1. Aqueous Oxygen alone: The AO is infused into the distal aorta, as described above. Oxygen is dissolved in water at a pressure of 1500 psi, and the material is infused by laminar flow through a narrow gauge intravenous catheter at a flow rate of 0.5 ml/min, for a period of 60 min, twice a day for 15 days.

Group 2. Antibiotic alone: The skin is treated with Cleocin T Gel, a topical formulation of the antibiotic clindamycin twice a day for a total of 15 days.

Group 3. Combined topical antibiotic and intra-arterial infusion of AO, as per above.

Group 4. Placebo: an infusion of normal saline, at the same pressure and pH as the AO; and topical administration of a placebo in lieu of the antibiotic, using the same base vehicle as the one into which the antibiotic is incorporated.

Directions:

In all cases, each time the topical preparation (whether placebo or active) is applied to the site of the infected ischemic skin, the lesion is then covered afresh with sterile gauze which is lined on the skin side with an impermeable layer known to not absorb the base vehicle of the antibiotic formulation. The bandage is secured in such a manner that the animal cannot pull or chew it off, and therefore cannot lick off the medication/placebo.

Step 3. Preparation of Specimen for Analysis:

The animals from all four groups are sacrificed on day 15, by i.v. injection of EuthanylR (pentobarbital sodium), 100-240 mg/kg. Within 5 min of sacrifice, the ischemic skin lesion is removed surgically by making an incision approximately ¼ of an inch wider than the circumference of the lesion, and that runs down to the fascial layer separating the dermis from the underlying muscle. The horizontal plane of the skin lesion is divided by scalpel cuts into four regions roughly equal in area, designated sections A, B, C and D, where sections A and D are the regions on the periphery of the lesion, and sections B and C are the regions in the middle of the lesion. Sections A and C are gently macerated without heating, the macerate then being suspended in 0.5 cc of normal sterile saline, which is then poured onto a petri dish containing the appropriate types and amounts of nutrients for growth of the infecting bacteria. The petri dish is then incubated for 48 hours at 37 degrees centigrade. Sections B and D are cut by vertical scalpel slices into smaller strips approximately ⅛ inch wide, which are mounted histologically for observation under a light microscope. The sections are then graded by an expert blinded for the conditions of the experiment who scores each on a scale ranging from complete necrosis to complete healing (measured as % of normal thickness of the epidermis, among other variables).

Step 4. Results:

Bacterial Counts:

From each of the four experimental armns, counts are made of the cfu of the bacteria grown from the macerated skin suspension that had been spread on the petri dish.

Histology:

From each of the four experimental arms, measurement are taken for skin thickness (representing healing of the lesion) and for the approximate number of inflammatory cells (PMNs, etc.) per cubic millimeter of skin necropsied.

These experiments show that the combination of oxygenating agent and antibiotic is more effective in reducing the bacterial counts than the antibiotic alone. Additionally, the combination shows a greater increase in skin thickness representing improved healing. There is also a greater decrease in the number of inflammatory cells with the use of the combination when compared with the antibiotic alone.

### Example 4

### Peritonitis: Use of Oxygenating Agent and/or Antibiotic Administered Parenterally, in the Treatment of Peritoneal Abscess (an Example of a Deep Infection where the Tissue Involved is Inherently Subject to Low Oxygen Tensions)

A mouse model of peritonitis described in the art is used (Onderdonk, A. B. et. al., “Use of a Model of Intraabdominal Sepsis for Studies of the Pathogenicity of Bacteroides fragilis”). The advantages of the mouse model are that, in order to induce peritoneal abscess formation (i) the bacterial inoculum requires the presence of only one bacterial species (Bacteriodes fragilis) plus caecal contents, and (ii) the inoculum can be injected directly into the peritoneal cavity, eliminating the need for surgical implantation.

Step 1: Establishing the Infection

Stock cultures of the obligate anaerobe Bacteroides fragilis (ATCC 23745), which is known in the art to promote abscess formation, are grown in pre-reduced peptone-yeast-glucose (PYG) (Scott-Robbins Laboratories, Fiskeville, R.I.) at 37° C. in an anaerobic chamber. After 18 hr, the culture is quick-frozen with liquid N₂ and stored at −60° C. until use.

Stock cultures of autoclaved mouse caecal contents are prepared for co-inoculation, serving as an adjuvant known in the art to ensure abscess formation. Caecal contents from 40 grain-fed mice are collected and pooled after killing by 100% CO₂. PYG broth is added to this material in order to attain a total volume 4-fold above that of the pooled caecal contents alone. The resultant slurry is filtered into a second beaker through 2 layers of coarse surgical gauze to remove large articulate matter. The final mixture is autoclaved at 121° C. for 2 hr and frozen at −60° C. until used.

To prepare the inoculum for injection, the frozen broth cultures of bacteria (10⁶ cfu/ml) and the frozen autoclaved mouse caecal contents are thawed in an anaerobic chamber. Equal volumes are thoroughly mixed in sterile tubes inside the chamber, and 1.0 ml amounts of this mixture are drawn into tuberculin syringes. These are capped with 18 gauge needles, and are removed from the chamber for immediate injection into the mice. 0.25 ml of the inoculum is then injected i.p, through the left side of the abdominal wall, without anesthesia.

Step 2. Treatment Modalities:

The animals are assigned to one of four groups, as described below. Treatment is delayed until there is objective evidence of peritonitis (fever, ruffled fur, exudate around the eyes, hunchback, etc.). In each group except the 4th, the respective treatment is administered intraarterially, once a day for a total of three consecutive days, according to the following method: A catheter placed in the left femoral artery is threaded anteriorly until it reaches (as demonstrated radiographically with dye injection) the ascending aorta, just below the level of the left brachial artery. In this manner, any material injected will be distributed by the arteries serving the abdominal cavity and the omentum, such that the oxygen carried by the oxygenating agent can diffuse out into the peritoneal cavity and thereby raise the pO₂ in the cavity. Prior to treatment, the exterior aspect of the left thigh is shaved and depilated by Scholl's Hair Remover (Scholl, Inc., Chicago, Ill.), and the area is prepared with iodine. Just prior to treatment, the animals are anesthetized by i.p. injection of 0.15 ml of Nembutal (50 mg/ml; Abbott, North Chicago, Ill.) and anesthesia is maintained throughout the ½ hr period of infusion.

Group 1: Treatment with the oxygenating agent FLUOSOL® alone. 0.5 mil of the FLUOSOL® is administered intraarterially over the course of 30 min, such that the aggregate dose of the drug administered totals 1.8 g per Kg body weight. This treatment is repeated at 2 h hr intervals for a total of 3 treatments.

Group 2 Intraarterial treatment with an antibiotic alone, namely clindamycin 150 mg/mL (it having been previously confirmed that the antibiotic is bactericidal for the strain of B. fragilis being used). The antibiotic is administered by slow infusion over a period of 30 min, such that the aggregate dose of the drug administered totals 600 mg.

Group 3: Combined intraarterial treatment with antibiotic and PERFLUBRON®, as per the above.

Group 4: Direct i.p. injection of FLUOSOL®. In this case 0.5 ml of FLUOSOL® emulsion is injected directly into the peritoneal cavity, on the side contralateral to the site of bacterial inoculation.

Group 5: Placebo: The animals receive the slow intraarterial infusion of the emulsion in which the FLUOSOL® would otherwise be contained, suspended in sterile normal saline, administered over the course of 30 min at a rate that will deliver the same amount of fluid as received by the animals in the other experimental arms.

Step 3. Results

Half the animals in each group are sacrificed 24 hours after the 3^{rd} and last treatment, and the other half are sacrificed 72 hours afterward, by breathing 100% CO₂. However, prior to sacrifice, and throughout the experiment, the animals are observed by raters.

1. “Clinical rating”: The animals are rated twice daily according to the following objective:
scale of visible outward signs of illness: 5=Normal in appearance; 4=Slightly ill (lethargy); 3=Moderately ill (lethargy, ruffled fur); 2=Critically ill (lethargy, ruffled fur, hunchback, exudate around the eyes); 4=Moribund; and 5=Dead.

2. Bacterial colony counts in peritoneal exudate and in the abscess (post-mortem):
For culture to determine quantitative bacterial counts in the purulent exudate, the abscesses are incised by aseptic techniques. An aliquot of 1.0 ml of purulent material is removed, added to 9.9 ml of prereduced VPI dilution salts, and transferred immediately to the anaerobic chamber. The specimen is homogenized with a tissue grinder, serial 100-fold dilutions are made, and 0.1 ml of each dilution is plated on prereduced brucella blood base agar. Colonies are counted after incubation for 48 hr, and results are expressed as cfu/ml pus.

3. Histopathology of the abscess (post-mortem).
Abscesses are removed by dissection from the peritoneal cavity, and are processed in two ways:

For histological section they are immediately placed in 20 ml of 10% formalin for 48-72 hr and are processed as follows: They are sectioned along the midline at the greatest diameter, in a plane perpendicular to the skin, producing two equal halves. One of the halves is again transected though the midline, but at 90° to the original section, resulting in two quarter-sections. Using either quarter-section, the distance from the exact center of the abscess to the external border of the lesion, in the plane of the skin and along the axis of second transections, is measured with calipers and recorded as “Radius 1”. The other half from the original hemisection is processed differently: this half is cut again in a plane parallel to the original cut and at the periphery of the abscess. The radius o this hemisphere along the same axis as Radius 1 is measured with calipers and recorded as Radius 2. The diameter of the abscess in a plane perpendicular to the histological section is equal to Radius 1+Radius 2.

The second hemisection is stained with haematoxylin and eosin and with aniline blue (collagen stain) for histological assessment. The stained sections are evaluated by light microscopy. Cross-sectional abscess area is measured by planimetry. Histological sections are magnified 4-fold with an enlarging lens (Schneider-Kreuznach 5.6/135), and the magnified image is projected on a frosted glass plate. Planimetry measurements are made with a Grafpen sonic digitizer (Design Data, Inc., Cambridge, Mass.) and a Hewlitt Packard 9830A digital computer. Abscess volume is calculated by the following formula:

$$Abscess volume = Cross \text{-} sectional abscess area on histological section \times \frac{Radius 1 + Radius 2}{2}$$

The experiment indicates that the combination of oxygenating agent and antibiotic is more efficacious in reducing the bacterial counts than the antibiotic alone. The combination also provides improved histopathology results.

### Example 5

### Pyorrhea: Topical Administration of an Oxygenating Agent and/or an Antibiotic to Control Pyorrhea in an Animal Model

Step 1.

Pyorrhea is established in the periodontal tissues of dogs, following the model described by Genco, C, Van Dyke, T, and Arnar, S. Animal models for Porphyromonas gingivalis-mediated periodontal disease. Trends in Microbiology, Vol. 6, No. 11, 1998.

Step 2. The Animals are Broken out Into Four Treatment Groups:

Group 1 receives once-daily applications of a topical formulation containing the liposomal perfluorocarbon agent A*O*C*S*®, in a base excipient known to diffuse into the periodontal space as well as to penetrate the superficial layers of the oral mucosa, said excipient thus enabling the oxygenating agent to reach the disease-causing bacteria in their hypoxic niche.

Group 2 receives once-daily topical applications on the gums of the antibiotic clindamycin in gel formulation (Cleocin T Gel 1%).

Group 3 receives once-daily combined treatments of both the FLUOSOL® and the antibiotic clindamycin.

Group 4 receives the excipients alone.

Step 3.

Ratings are made at intervals to determine the ability of the above-listed treatments to halt the progress of the disease. These rating are made by: (i) visual inspection of the appearance of the gums, and (ii) enumeration of the types and numbers of the bacterial species present in scrapings from the infected gums. At the end of the experiment, the animals are sacrificed humanely, and periodontal tissues are removed for histological analysis of the degree of infiltration of the various inflammatory cells (PMNs, etc.).

Step 4 Results:

The experiment further indicates the improvements obtainable when using the combination of oxygenating agent and antibiotic, e.g. the combination is more efficacious in reducing the bacterial counts than the antibiotic alone.

While the present invention has been described in connection with what is presently considered to be practical and preferred embodiments, it is understood that the present invention is not to be limited or restricted to the disclosed embodiments but, on the contrary, is intended to cover various modifications and equivalent arrangements included within the spirit and scope of the appended claims.

Thus, it is to be understood that variations in the described invention will be obvious to those skilled in the art without departing from the novel aspects of the present invention and such variations are intended to come within the scope of the claims below.

1. A method of treating a microbial infection in a subject comprising:
(a) providing an oxygenating agent that has been encapsulated in an encapsulating vehicle sufficient to prevent the oxygen from diffusing away from said agent until such time as the encapsulating vehicle has reached the site of infection; and
(b) administering an effective dosage of said encapsulated oxygenating agent to said subject.

2. The method of treating a microbial infection as claimed in claim 1, wherein said oxygenating agent is at least one member selected from the group consisting of (i) a blood substitute based on cell-free hemoglobin and heme products and (ii) encapsulated hemoglobin and heme products.

3. The method of treating a microbial infection as claimed in claim 2, wherein said oxygenating agent is at least one member selected from the group consisting of (a) synthetic heme compounds, wherein said synthetic heme compounds are modified heme compounds having an alkaneimidazole group binding iron at the proximal sixth coordination site and with four long-chain alkanephosphocholine groups whic provide lipophilicity and an oxygen pocket; (b) liposome-encapsulated hemoglobin preparations, which can function as artificial red blood cells; and (c) modified hemoglobins.

4. The method of treating a microbial infection as claimed in claim 3, wherein said oxygenating agent is selected from the group consisting of Pyridoxalated Hemoglobin Polyoxyethylene Conjugate (“PHP”); PEG-hemoglobin; o-Raffinose Poly Hemoglobin (“Hemolink”); Polynitroxyl-Hemoglobin (“PNH”); polymerized human hemoglobin (“Poly SFH—P”); polymerized purified bovine hemoglobin; and cross-linked hemoglobins.

5. The method of treating a microbial infection as claimed in claim 4, wherein said cross-linked hemogobin is Diaspirin Crosslinked Hemoglobin (“DCLHb”, HEMASSIST™).

6. The method of treating a microbial infection as claimed in claim 1, wherein said oxygenating agent is a non-hemoglobin, non-heme material that dissolves oxygen, said material comprising synthetic chemical compounds of the class of agents known as perfluorocarbons (PFCs).

7. The method of treating a microbial infection as claimed in claim 6, wherein said synthetic chemical compounds are selected from the group consisting of perfluorodecalin (C₁₀F₁₈), perfluoro-tri-n-proplyamine (C₉F₂₁N), fluoromethylo-adamantane (“FMA”), OXYGENT® (perfluoroctylbromide), PERFLUBRON® (C₈F₁₇Br), and FLUOSOL-DA®.

8. The method of treating a microbial infection as claimed in claim 1, wherein said oxygenating agent is a non-hemoglobin, non-heme material that dissolves oxygen, said material being water supersaturated with oxygen to form Aqueous Oxygen, said Aqueous Oxygen then being perfused for regional or systemic hyperoxemia.

9. The method of treating a microbial infection as claimed in claim 1, wherein said oxygenating agent generates oxygen by a chemical reaction.

10. The method of treating a microbial infection as claimed in claim 9, wherein said oxygenating agent is selected from the group consisting of hydrogen peroxide, tetrachlorodecaoxide, ozone, and potassium permanganate.

11. The method of treating a microbial infection as claimed in claim 1, wherein said subject is a human.

12. The method of treating a microbial infection as claimed in claim 1, wherein the oxygenating agent is delivered topically for intradermal or intramucosal penetration.

13. The method of treating a microbial infection as claimed in claim 12, wherein said oxygenating agent is formulated in pharmaceutical compositions to produce a formulation capable of penetrating the layers of the dermis or the mucosa.

14. The method of treating a microbial infection as claimed in claim 13, wherein said formulation is selected from the group consisting of penetrating emollients and carriers on transdermal patches.

15. The method of treating a microbial infection as claimed in claim 14, wherein said formulation is dimethylsulfoxide (“DMSO”).

16. The method of treating a microbial infection as claimed in claim 12, wherein said oxygenating agent is formulated in an encapsulated pharmaceutical composition or a microencapsulated pharmaceutical composition, said formulation penetrating the layers of the dermis or the mucosa.

17. The method of treating a microbial infection as claimed in claim 16, wherein said formulation is encapsulated in a carrier comprising a form composed of at least one member selected from the group consisting of lipids, amino acids, and polymers.

18. The method of treating a microbial infection as claimed in claim 17, wherein said form is selected from the group consisting of spheres, microspheres, cochlear shapes, and dendrimers

19. The method of treating a microbial infection as claimed in claim 18, wherein said microspheres comprise at least one member selected from the group consisting of natural polymers, proteins, carbohydrates and waxes.

20. The method of treating a microbial infection as claimed in claim 19, wherein said microspheres comprise at least one member selected from the group consisting of gelatin, albumin, casein, gum arabic, gum acacia, agar, alginates, carrageenan, starches, xanthan, beeswax and shellac.

21. The method of treating a microbial infection as claimed in claim 19, wherein said microspheres comprise at least one member selected from the group consisting of celluose esters, cellulose ethers, fatty acid derivatives and fatty alcohol derivatives.

22. The method of treating a microbial infection as claimed in claim 19, wherein said microspheres comprise at least one member selected from the group consisting of methyl cellulose, ethyl cellulose, cellulose acetate, celluolose acetate butyrate, sodium carboxymethly cellulose, cellulose nitrate, glyceryl-mono-, di-, or tri-stearate, stearic acid, aluminum monostearate, glyceryl mono- and di-palmitate, hydrogenated tallow, 12-hydroxy-stearyl alcohol, hydrogenated castor oil, cetyl alcohol, and myristyl alcohol (1-tetra-decanol).

23. The method of treating a microbial infection as claimed in claim 19, wherein said microspheres comprise synthetic polymers.

24. The method of treating a microbial infection as claimed in claim 23, wherein said synthetic polymer is selected from the group consisting of (a) vinyl polymers and copolymers, (b) polyamides and polyesters, (c) polymers prepared by interfacial polymerization, (d) waxes and resins and (e) amino resins, alkyd resins, epoxy-resins, polyester resins, polydimethyl siloxane, and polycarbonates.

25. The method of treating a microbial infection as claimed in claim 24, wherein said synthetic polymer is selected from the group consisting of polyvinyl alcohol, polyacrylamide and copolymers, ethylene-vinyl acetate copolymers, polymethyl methacrylate, polyvinyl pyrrolidone, polystyrene, styrene-acrylonitrile copolymers, polyvinylidiene chloride, vinyl ether coploymers, carboxyvinyl polymers (“Carbopol”), nylon 6-10, polylysine and copolymers, polyglutamic acid and copolymers, polylactic acid and copolymers, hydrogel polymers (polyhydroxyethyl methacrylate and copolymers), polyglycolic acid, polyurethanes, paraffin wax and hydrocarbon wax.

26. The method of treating a microbial infection as claimed in claim 19, wherein said microspheres comprise polymers that degrade in a predictable manner, wherein said time-released delivery of said oxygenating agents is controlled.

27. The method of treating a microbial infection as claimed in claim 26, wherein the microspheres comprise poly-lactide-co-glycolide, said microspheres being administered to the subject as a subcutaneous implant.

28. The method of treating a microbial infection as claimed in claim 1, wherein said oxygenating agent is delivered by a needle-injection method selected from the group consisting of subcutaneously; directly into a superficial infection (such as a boil) or into a deep infection (such as an intra-abdominal abscess); intramuscularly; intravenously; intra-arterially; intracardiac; intrapericardiac; intrathecal; by lumbar puncture; or by burr hole directly onto the meninges or into the parenchyma of the brain.

29. The method of treating a microbial infection as claimed in claim 1, wherein said oxygenating agent is delivered a delivery method selected from the groups consisting of (a) by intravascular catheter for perfusion of the large vessels of an organ, for example in order to perfuse an infarcted and infected bowel segment; (b) instillation tube, for example for instillation into the abdominal cavity in the case of an abscess; (c) by catheter to irrigate the lumen of a hollow organ (such as the urinary bladder and the uterus), or the lumen of the gastrointestinal tract (some segments of which provide an anaerobic or microaerophilic milieu that favors the multiplication of pathogens, as for example in Helicobacter pylori infections of the stomach or duodenum and the ulcers concomitant therewith, and Crohn's disease and/or ulcerative colitis, these being conditions well known to be controlled with the prescription of various antibiotics); and (d) by endoscopic instrument, for example for irrigation of (i) the fallopian tubes or of (ii) the apical segments of the lung (these segments being relatively poorly oxygenated and therefore prone to low-level colonization by Mycobacterium tuberculosis).

30. The method of treating a microbial infection as claimed in claim 1, wherein said oxygenating agents are incorporated into bandages, dressings and/or packings, (i) for superficial or deep wounds, (ii) for incisions, (iii) for fistulae, and/or (iv) for the prophylactic protection of areas of the skin that are prone to develop or are already developing pressure sores, gangrene, or cellulitis.

31. The method of treating a microbial infection as claimed in claim 1, wherein said oxygenating agents are incorporated into toothpastes, gels and/or packings that can be used by the patient or inserted by the dentist, in the treatment and prevention of periodontal disease and the bone loss concomitant therewith.

32. The method of treating a microbial infection as claimed in claim 1, wherein said infecting microbe is any type of bacterium, virus, yeast, fungus, mold, algae or parasite (protozoa, amoeba, or other form).

33. The method of treating a microbial infection as claimed in claim 1, wherein said infecting microbe can be from any group, but is particularly one generally recognized as being difficult to treat, therefore rendering the infected human or animal in need of an adjunctive treatment such as that of the present invention.

34. The method of treating a microbial infection as claimed in claim 1, wherein said microbe is difficult to treat by virtue of its location being a hypoxic/ischemic site, which condition results in a decreased rate of microbial replication, thereby rendering said locus of microbes unresponsive to those antimicrobial agents that require active replication for effectiveness.

35. The method of treating a microbial infectiona as claimed in claim 34, wherein said microbe is selected from the group consisting of S. aureus, P. aeruqinosa, S. typhimurium, and M. tuberculosis.

36. The method of treating a microbial infection as claimed in claim 1, wherein said microbe is difficult to treat by virtue of its being in a location into which it is difficult for antimicrobial agents to diffuse.

37. The method of treating a microbial infection as claimed in claim 36, wherein said location is selected from the group consisting of intracellular locations such as macrophages, T cells, neurons and hepatocytes; any walled-off area, such as an abscess or a tubercle; inside a body cavity; inside a sac (such as the pericardium); inside a joint; in the recesses of bone; in the lumen of a hollow organ (e.g., the gastrointestinal tract, urinary bladder, genital organs, and upper and lower respiratory tract and the sinuses thereof; in a periodontal site; or in the linings of any organ (e.g., peritoneum, pleural lining, and the meninges or other linings of the brain).

38. The method of treating a microbial infection as claimed in claim 1, wherein said microbe is difficult to treat by virtue of its being a strain that is multidrug resistant.

39. The method of treating a microbial infection as claimed in claim 38, wherein said microbe is selected from the group consisting of vancomycin-resistant Enterococcus faecium and vancomycin intermediate-resistant Staphylococcus aureus.

40. The method of treating a microbial infection as claimed in claim 1, wherein said microbe is one that can be directly harmed by increases in pO₂.

41. The method of treating a microbial infection as claimed in claim 40, wherein said microbe is selected from the group consisting of Clostridium difficile, Clostridium perfringens, Propionibacterium acnes and Porphyromonas gingivalis.

43. The method of treating a microbial infection as claimed in claim 1, wherein said microbe is one that can be indirectly harmed by increases in pO₂, in that said increase will augment the innate host antimicrobial defenses.

44. The method of treating a microbial infection as claimed in claim 43, wherein said augmentation is an increased ability under a higher pO₂ of various white blood cells to generate free radicals that will in turn kill intracellular specimens of (i) S. typhimurium and of (ii) the Human Immunodeficiency Virus.

45. The method of treating a microbial infection as claimed in claim 1, further comprising adjunctive synergistic agents, singly or in various combinations or permutations, being co-administered with the oxygenating agent, either together in a pharmaceutical co-formulation, or separately in time and space.

46. The method of treating a microbial infection as claimed in claim 45, wherein said adjunctive synergistic agent is an antimicrobial agent appropriate to the infecting microbe.

47. The method of treating a microbial infection as claimed in claim 46, wherein said antimicrobial agent is an antibacterial agent.

48. The method of treating a microbial infection as claimed in claim 47, wherein said antibacterial agent is one selected from the group consisting of antibiotics; chemotherapeutic agents (such as sulfamethoxazol); bacteriophages and defective bacteriophages; bacteriocins and bacteriocin-like substances (“BLSs”); defensins and all related peptide-based antibacterial agents; therapeutic antibodies or vaccines administered acutely to treat an infection; and sterilizing agents and disinfecting agents.

49. The method of treating a microbial infection as claimed in claim 48, wherein said bacteriophages, which cannot efficiently propagate (if at all) when the bacterial target is in a hypoxic milieu (and is therefore not dividing), said bacteriophages will be made more efficient by the present invention, whether they be wild-types, or strains patented on account of special characteristics (such as long circulation time and therefore delayed clearance by the RES).

50. The method of treating a microbial infection as claimed in claim 47, wherein said antimicrobial agent is specific for viruses, said antimicrobial agent being selected from the group consisting of AZT, Zovirax, and interferons.

51. The method of treating a microbial infection as claimed in claim 47, wherein said antimicrobial agent is specific for yeasts, said agent being selected from the group consisting of Nystatin and Vagistat.

52. The method of treating a microbial infection as claimed in claim 47, wherein said antimicrobial agent is specific for fungi, said agent being selected from the group consisting of Amphotericin B, abelcet, and ketoconazole.

53. The method of treating a microbial infection as claimed in claim 47, wherein said antimicrobial agent is specific for parasites, whether unicellular or multicellular, said agent being selected from the group consisting of Flagyl, iodoquin, quinine and atabrine.

54. The method of treating a microbial infection as claimed in claim 45, wherein said adjunctive synergistic agent is an antioxidant, said antioxidant being selected from the group consisting of tocopherol (vitamin E), catalase, ascorbic acid and superoxide dysmutase (“SOD”).

55. The method of treating a microbial infection as claimed in claim 45, wherein said adjunctive synergistic agent is an endotoxin antagonist, said antagonist being selected from the group consisting of monoclonal antibodies or derivatives of reconstituted high density lipoproteins (“rHDL”).

56. The method of treating a microbial infection as claimed in claim 45, wherein said adjunctive synergistic agent is a cytokine modulator that attenuates the deleterious effects of the infecting microbes and their toxins.

57. The method of treating a microbial infection as claimed in claim 45, wherein said adjunctive synergistic agent is a growth factor that can help the repair and healing of a tissue, said agent being selected from the group consisting of epithelial growth factors (EGFs) and would also include interferons, cytokines, chemokines, and MHC type 2-inducing or -modulating factors.
